(19) United States
(12) Patent Application Publication
(10) Pub. No.: US 2017/0333390 A1
(43) Pub. Date: Nov. 23, 2017

(54) PHYTOCHEMICAL COMPOSITIONS INCLUDING SESAMIN FOR ANTI-INFLAMMATORY, ANTI-CYTOKINE STORM, AND OTHER USES

(71) Applicants: CHIANG MAI UNIVERSITY, Chiang Mai (TH); COMMISSION ON HIGHER EDUCATION, Bangkok (TH)

(72) Inventor: Prachya KONGTAWELERT, Chiang Mai (TH)

(21) Appl. No.: 15/622,639

(22) Filed: Jun. 14, 2017

Related U.S. Application Data
(62) Division of application No. 14/561,928, filed on Dec. 5, 2014.
Application No. 14/561,928 is a division of application No. 13/496,189, filed on Mar. 14, 2012, now abandoned.

(51) Int. Cl. A61K 31/36; A23L 33/105; A61K 45/06

(52) U.S. Cl. CPC A61K 31/36; A61K 45/06; A23L 33/105; A23V 2002/00

(57) ABSTRACT

A composition (e.g., phytochemical composition) including sesamin, the composition exhibiting anti-inflammatory, anti-cytokine storm, connective tissue preservation, anti-viral, and/or other properties in biological tissue. The phytochemical composition therapeutically affects a pro-inflammatory cytokine condition, for instance facilitating or effectuating decrease in a quantity of a pro-inflammatory cytokine, for example, interleukin-1 and tumor necrosis-alpha. The phytochemical composition facilitates or effectuates an increase in quantity of an anti-inflammatory cytokines. The phytochemical composition can additionally facilitate a connective tissue extracellular matrix (ECM) preservation effect. The phytochemical composition can also inhibit an action of a viral neuraminidase, for example, the influenza A virus neuraminidase. Uses of sesamin for manufacture of phytochemical compositions having predetermined concentrations of sesamin include facilitating a decrease in quantity of pro-inflammatory cytokines, an increase in anti-inflammatory cytokines, a connective tissue anti-degenerative effect, and/or an inhibition of viral neuraminidase within the living organism. Manufacturing processes for the phytochemical compositions are also described.

### TECHNICAL FIELD

The present disclosure relates generally to phytochemical compositions that include sesamin or a sesamin-class compound. More specifically, the present disclosure relates to phytochemical compositions including sesamin or a sesamin-class compound that exhibit anti-inflammatory, anti-cytokine storm, and other uses.

### BACKGROUND

A body's immune system and immune cells are essential for fighting pathogens, for example bacteria, viruses, and other foreign matter, that invade or are introduced within the body. Recognition of pathogens within the body typically triggers an immune response. Generally, the immune response involves a production of cytokines. It is generally important that the production of cytokines is well regulated for maintaining homeostatic balance within the body. An imbalance in the production of cytokines, for example an excessive production of cytokines, in the body can cause significant damage to body tissues and organs.

A cytokine storm (which is also known as hypercytokinemia) is a significant immune response to pathogens that invade the body. The precise causation of cytokine storms within the body has not been definitively established. A possible causation of cytokine storms is an encounter, by the immune system, of a new and highly pathogenic pathogen. Cytokine storms are also associated with a number of infectious and non-infectious diseases, including influenza, adult respiratory distress syndrome (ARDS), and systemic inflammatory response syndrome (SIRS). It has been suggested that the influenza A (H1N1) virus triggers cytokine storms within the body.

During a cytokine storm, inflammatory mediators, for example pro-inflammatory cytokines such as Interleukin-1 (IL1), Interleukin-6 (IL6), tumor necrosis factor-alpha (TNF-alpha), oxygen free radicals, and coagulation factors are released by the immune cells of the body. Cytokine storms have the potential to cause significant damage to body tissues and organs. For example, occurrence of cytokine storms in the lungs can cause an accumulation of fluids and immune cells, for example macrophages, in the lungs, and eventually block off the body's airways thereby resulting in respiratory distress and even death.

It is generally important to be able to prevent, control, or mitigate the occurrence of the cytokine storm. There are existing or conventional techniques associated with preventing, controlling, or mitigating the occurrence of the cytokine storm (and unwanted inflammation in general). For example, it has been reported that TNF inhibitors may be useful for facilitating the control of cytokine storms and for reducing adverse reactions caused by the occurrence of cytokine storms within the body. In addition, research has suggested that angiotensin converting enzyme (ACE) inhibitors, and angiotensin II receptor blockers (ARBs), may have clinical utility for controlling or down-regulating cytokine storms, and for reducing inflammation, within the body. Corticosteriods and non-steriodal anti-inflammatory drugs (NSAIDS) have also been employed in an attempt to treat patients experiencing cytokine storms. In addition, suggested therapeutic agents for treating influenza-type viral infections include antibodies to the influenza virus neuraminidase.

However, the effectiveness and safety of many conventional techniques associated with the prevention, control, or mitigation of cytokine storms within the body have not been comprehensively or adequately verified. In addition, many conventional anti-influenza treatments (e.g. anti-viral or anti-influenza drugs) have associated undesirable side effects when consumed by a patient. Such undesirable side effects include nausea, vomiting, and toxicity. Furthermore, there is an increasing problem of developed resistance to many conventional anti-viral (e.g. anti-influenza) drugs.

Accordingly, new or enhanced approaches for at least one of preventing, controlling, or mitigating cytokine storms may be useful for improving public health. Furthermore, new compositions or techniques providing anti-influenza or anti-inflammatory properties may also be useful for improving public health.

### SUMMARY

In accordance with a first aspect of the present disclosure, there is disclosed a composition comprising a predetermined concentration of sesamin to provide a sesamin dosage of at least approximately 5 mg/day when consumed by a living organism. The composition at least one of facilitates and effectuates decrease in a quantity of a pro-inflammatory cytokine within the living organism when consumed thereby.

In accordance with a second aspect of the present disclosure, there is disclosed a use of sesamin for the manufacture of a composition that comprises sesamin to therapeutically affect a pro-inflammatory cytokine condition within a living organism when consumed thereby, the composition providing a sesamin dosage of at least approximately 5 mg/day when consumed by the living organism.

In accordance with a third aspect of the present disclosure, there is disclosed a process for manufacturing a composition comprising sesamin that that at least one of facilitates and effectuates a decrease in quantity of a pro-inflammatory cytokine within a living organism when consumed thereby. The process comprises providing the composition with a sesamin concentration that provides a living organism with a sesamin dosage of at least approximately 5 mg/day when consumed thereby.

In accordance with a fourth aspect of the present disclosure, there is disclosed a composition comprising a concentration of sesamin to provide a daily sesamin dosage of at least approximately 5 mg/day when consumed by a living organism and at least one of an anti-inflammatory substance, a connective tissue anti-degradation substance, an anti-viral drug, and a cholesterol lowering substance.

In accordance with a fifth aspect of the present disclosure, there is disclosed a composition comprising sesamin of a concentration of at least one of approximately 1.0 mg/L and approximately 1.0 mg/kilogram, the composition at least one of facilitating and effectuating decrease in a quantity of a pro-inflammatory cytokine within a living organism when consumed thereby.

In accordance with a sixth aspect of the present disclosure, there is disclosed a use of sesamin for the manufacture of a composition that comprises at least one of approximately 1.0 mg/L and approximately 1.0 mg/kilogram of sesamin to therapeutically affect a pro-inflammatory cytokine condition within a living organism when consumed thereby.

### BRIEF DESCRIPTION OF THE DRAWINGS

FIG. 1 is a diagram showing example effects of sesamin concentrations of approximately 0.025 mM, 0.05 mM, 0.1 mM, 0.25 mM, 0.50 mM and 1.0 mM on IL-1 beta expression relative to GAPDH in LPS induced synovial fibroblast cells.

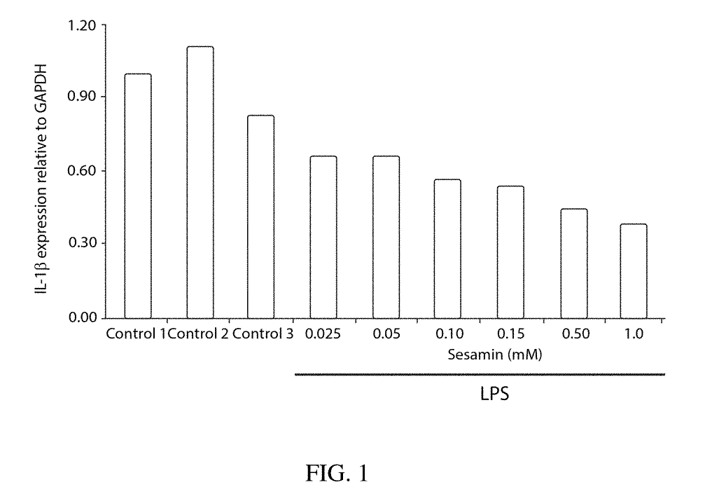

FIG. 2. is a diagram showing example effects of sesamin concentrations of approximately 0.1 uM, 0.5 uM, 1.0 uM, 5.0 uM, and 10.0 uM on quantity of IL-1beta released by LPS induced peripheral blood mononuclear cells (PBMCs).

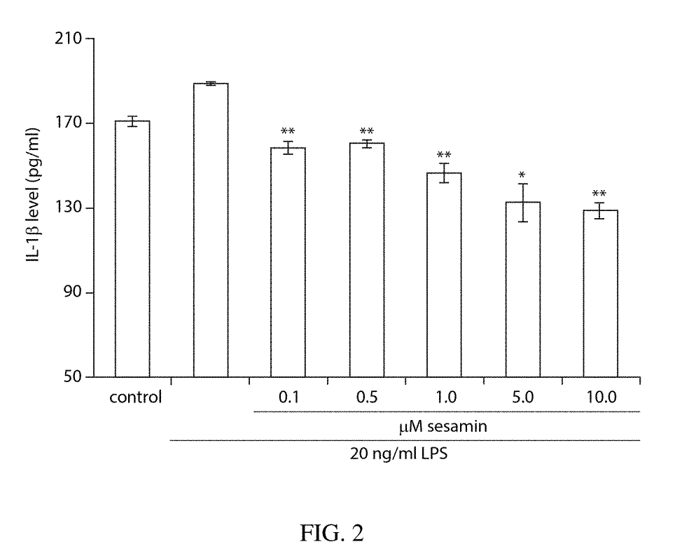

FIG. 3 is a diagram showing example effects of sesamin concentrations of approximately 0.1 ug/ml, 0.5 ug/ml, 1.0 ug/ml, and 5.0 ug/ml on IL-2 gene expression in peripheral blood mononuclear cells (PBMCs).

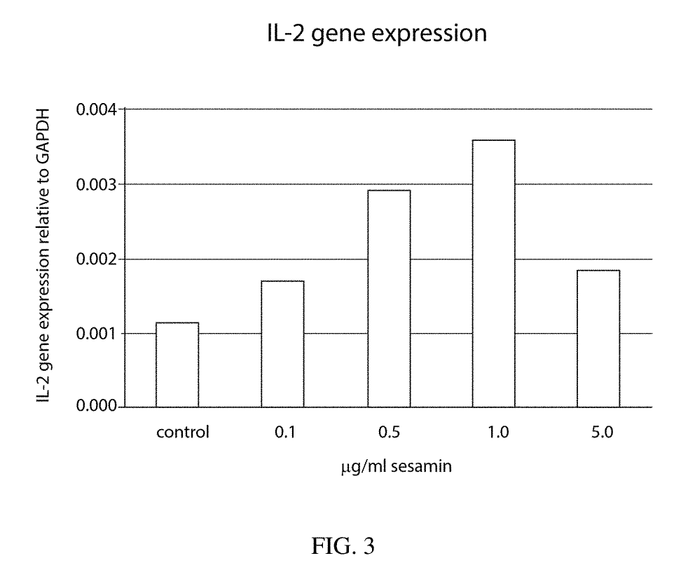

FIG. 4. is an example computer graphic simulation of binding of a sesamin molecule to the influenza A (H1N1) virus and the binding energy of this bond.

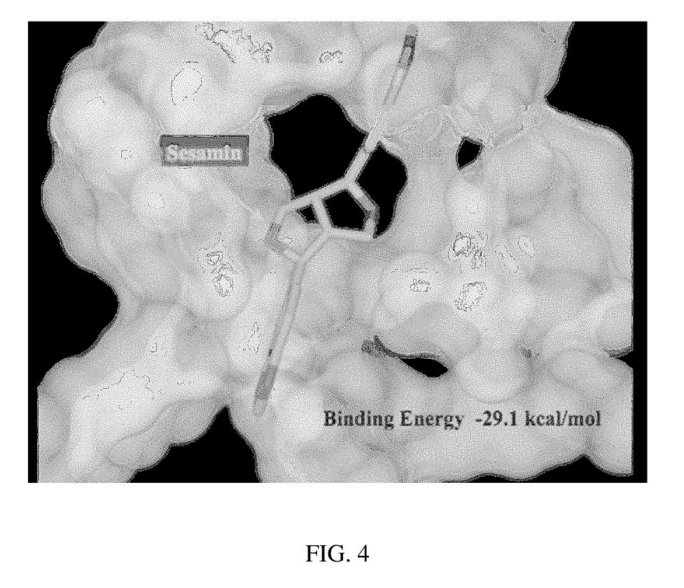

FIG. 5 is a diagram showing example effects of sesamin concentrations of approximately 0.5 ug/ml, 1.0 ug/ml and 10.0 ug/ml on quantity of IL-1 pg/ml released.

FIG. 6 is a diagram showing example effects of sesamin concentrations of approximately 0.5 ug/ml, 1.0 ug/ml and 10.0 ug/ml on percentage of quantity of IL-1 released relative to isolated egg cells (diluted in a ratio of 1:50).

### DETAILED DESCRIPTION

A cytokine storm (also known as hypercytokinemia) is a significant immune response within the body that typically occurs during the course of a number of infectious and non-infectious diseases, for example influenza and acute respiratory distress syndrome. During the cytokine storm, a number of pro-inflammatory cytokines and pro-inflammatory mediators, for example interleukin-1, interleukin-6, interleukin-8, and tumor necrosis factor-alpha, are produced and released by immune cells within the body. Cytokine storms have the potential to cause significant damage to body tissues and organs. The effectiveness and safety of many conventional compositions, methods, and/or processes used for preventing, controlling, down-regulating, and/or stopping cytokine storms within the body have not been comprehensively or adequately verified. Many conventional compositions providing anti-viral (e.g., anti-influenza) or anti-inflammatory effects have associated undesirable side effects when consumed by a patient. Such undesirable side effects include nausea, vomiting, and toxicity. In addition, there is an increasing problem of the development of resistance to many conventional anti-viral (e.g., anti-influenza) drugs. Accordingly, there is a need for new compositions for facilitating the prevention, control, down-regulation, and/or termination of cytokine storms, including compositions supporting, facilitating, or having anti-viral (e.g., anti-influenza) or anti-inflammatory effects or uses, and which reduce or minimize a likelihood that undesirable side effects or the development of resistance will occur.

Embodiments of the present disclosure are directed to compositions (e.g., phytochemical compositions) that include sesamin, methods and processes for manufacturing said compositions (e.g., phytochemical compositions), and uses of sesamin for the manufacture of said compositions (e.g., phytochemical compositions). In the context of the present disclosure, the term phytochemical shall refer to any compound or chemical that occur naturally in plants (i.e. living organisms belonging to the kingdom Plantae), or to any artificial or synthetic compound, substance, or chemical having an identical, almost identical, or significantly similar chemical, physical, and/or structural properties to that which occurs naturally in plants, and the term phytochemical composition shall refer to a composition including at least one phytochemical. Unless defined otherwise, all technical and scientific terms used herein have the same meanings as commonly understood by a person of ordinary skill in the relevant art of the present disclosure.

Most embodiments of the present disclosure are directed to compositions (e.g., phytochemical compositions) that include predetermined concentrations of sesamin for therapeutically affecting a pro-inflammatory cytokine condition, for instance by facilitating or effectuating a decrease or reduction in a quantity of pro-inflammatory cytokines or pro-inflammatory mediators within biological tissue (e.g., a body of a living organism when consumed thereby). In the context of the present disclosure, the term living organism refers to human beings and animals (i.e., organisms from the kingdom Animalia).

Examples of pro-inflammatory cytokines or pro-inflammatory mediators include interleukin-1alpha (IL-1α) and interleukin-1beta (IL-1β) (hereinafter collectively referred to as interleukin-1 or IL-1), interleukin-6 (IL-6), interleukin-8 (IL-8), interleukin-11 (IL-11), interleukin-12 (IL-12), interleukin-17 (IL-17), interleukin-18 (IL-18), tumor necrosis factor-alpha (TNF-α), interferon-gamma (IFN-γ), granulocyte-macrophage colony stimulating factor (GM-CSF), and transforming growth factor-beta (TGF-β). It will be understood by a person having ordinary skill in the art that references to pro-inflammatory cytokines in most embodiments of the present disclosure can refer any one or more of pro-inflammatory cytokines that are known in the art, including the above-listed examples of pro-inflammatory cytokines.

In some embodiments, the decrease in quantity of pro-inflammatory cytokines within the living organism helps to prevent, control, down-regulate, and/or stop occurrence of a cytokine storm within the living organism. This is to say, in some embodiments, the phytochemical composition facilitates or provides an anti-cytokine storm effect or function when consumed by the living organism. In several embodiments, the decrease in quantity of pro-inflammatory cytokines within the living organism helps to prevent, control, down-regulate, and/or stop inflammation within the living organism. This is to say, in several embodiments, the phytochemical composition facilitates or provides an anti-inflammatory effect or function when consumed by the living organism. In numerous embodiments, the decrease in quantity of pro-inflammatory cytokines within the living organism contributes to an anti-viral (e.g., anti-influenza) effect or function within the living organism.

Some embodiments of the present disclosure are directed to phytochemical compositions that include predetermined concentrations of sesamin for therapeutically affecting an anti-inflammatory cytokine condition, for instance by facilitating or effectuating an increase in anti-inflammatory cytokines, anti-inflammatory mediators, and/or anti-inflammatory factors within the living organism. Examples of anti-inflammatory cytokines, anti-inflammatory mediators, and/or anti-inflammatory factors include interleukin-2 (IL-2), interleukin-4 (IL-4), interleukin-13 (IL-13), and interferon-alpha (IFN-α). A person having ordinary skill in the art will understand that a reference to anti-inflammatory cytokines, anti-inflammatory mediators, and/or anti-inflammatory factors in most embodiments of the present disclosure can relate to any one or more of anti-inflammatory cytokines, anti-inflammatory mediators, and/or anti-inflammatory factors that are known in the art, including the above-listed examples.

Some embodiments of the present disclosure are directed to phytochemical compositions that include predetermined concentrations of sesamin for facilitating or effectuating inhibition of a viral neuraminidase (e.g., a decrease in an action of viral neuraminidase). In numerous embodiments, the viral neuraminidase is a neuraminidase of a virus contributing to occurrence of cytokine storms within the body of the living organism. In several embodiments, the viral neuraminidase is a neuraminidase of an influenza virus (e.g., influenza virus A, influenza virus B, and influenza virus C). In selected embodiments, the viral neuraminidase is an influenza A (H1N1) neuraminidase virus. In other embodiments, the viral neuraminidase is an influenza A (H2N1), influenza A (H2N2), influenza A (H3N2), influenza A (H5N1), or influenza A (H1N2) neuraminidase virus.

Selected embodiments of the present disclosure are directed to phytochemical compositions that include predetermined concentrations of sesamin for facilitating or effectuating any combination of one or more of a decrease or reduction in a quantity of pro-inflammatory cytokines, an increase in quantity of anti-inflammatory cytokines, and an inhibition of a viral neuramidase (e.g., the influenza A neuraminidase virus) within the living organism.

Several embodiments of the present disclosure are directed to phytochemical compositions that include predetermined concentrations of sesamin for facilitating or effectuating at least one of prevention, control, down-regulation, or termination of cytokine storms caused or triggered by viral infections (e.g., infection by the influenza viruses, including influenza A (H1N1), influenza A (H2N2), and influenza A (H2N3) viruses).

In some embodiments of the present disclosure, the phytochemical composition facilitates a decrease in the gene expression of the pro-inflammatory cytokine by an immune cell. In several embodiments, the phytochemical composition facilitates decrease in the gene expression of at least one of IL-1 and TNF-α by the immune cell. Immune cells refer to cells that participate or are involved in an immune response within the body. Examples of immune cells include white blood cells (e.g., leukocytes), which includes phagocytes, macrophages, neutrophils, dendritic cells, mast cells, eosinophils, basophils, and natural killer cells, and lymphocytes. In some embodiments, the phytochemical composition facilitates a decrease in release or secretion of the pro-inflammatory cytokine from the immune cell.

In some embodiments of the present disclosure, the phytochemical composition facilitates an increase in gene expression of anti-inflammatory cytokines by the immune cell. In selected embodiments, the phytochemical composition facilitates an increase in gene expression of IL-2 by the immune cell. In several embodiments, the phytochemical composition facilitates an increase in release or secretion of the anti-inflammatory cytokine from the immune cell.

In many embodiments of the present disclosure, the concentration of sesamin of the phytochemical composition can be varied as required. In many embodiments, the concentration of sesamin within the phytochemical composition is selected or determined for providing a living organism with a sesamin dosage of at least approximately 5 milligrams (mg) per day when consumed by the living organism. In some embodiments, the concentration of sesamin of the phytochemical composition is selected or determined for providing the living organism with a sesamin dosage of at least 10 mg per day when consumed by the living organism. In several embodiments, the concentration of sesamin of the phytochemical composition is selected or determined for providing the living organism with a sesamin dosage of at least 20 mg per day when consumed by the living organism.

In many embodiments of the present disclosure, the phytochemical composition has a concentration of sesamin of substantially between approximately 0.01 mM and approximately 2.0 mM. In some embodiments, the phytochemical composition has a concentration of sesamin of substantially between approximately 0.025 mM and approximately 1.0 mM. In selected embodiments, the phytochemical composition has a concentration of sesamin of substantially between approximately 0.10 mM and approximately 0.50 mM.

In many embodiments of the present disclosure, the phytochemical composition is or forms a portion of one of a food product, a beverage product, and a drug or pharmaceutical product. In other embodiments of the present disclosure, the phytochemical composition is a supplement, additive, or ingredient for one of a food product, a beverage product, and a drug, pharmaceutical, or other consumable (e.g., ingestible) product. It will be understood by a person having ordinary skill in the art that the phytochemical composition can be in various forms or formulations, for example a powder, a paste, or an emulsion.

When particular phytochemical compositions of the present disclosure form portions of beverage products, sesamin concentrations of such phytochemical compositions can be varied as required to correspondingly vary the sesamin concentrations of the beverage products. In some embodiments, the sesamin concentration in the beverage product is at least approximately 1.0 mg/liter of the beverage product. In other embodiments, the sesamin concentration in the beverage product is at least approximately 5.0 mg/liter or approximately 10 mg/liter of the beverage product.

When particular phytochemical compositions of the present disclosure form portions of food or other consumable products, sesamin concentrations of such phytochemical compositions can be varied as required to vary the sesamin concentration of the food or other consumable products. In some embodiments, the sesamin concentration in the food or other consumable product is at least approximately 1.0 mg/kilogram of the food or other consumable product. In other embodiments, the sesamin concentration in the food or other consumable product is at least approximately 5.0 mg/kilogram or approximately 10 mg/kilogram of the food or other consumable product.

In some embodiments of the present disclosure, particular phytochemical compositions as disclosed above can be combined, mixed, synthesized, or manufactured with other drugs, pharmaceutical compositions, phytochemical compositions, and/or nutraceutical compositions. In several embodiments, the phytochemical compositions as disclosed above can be combined, mixed, synthesized, or manufactured with anti-viral (e.g., anti-influenza) and/or anti-inflammatory substances, drugs, pharmaceutical compositions, phytochemical compositions, and/or nutraceutical compositions. In selected embodiments, the phytochemical compositions as disclosed above can be combined, mixed, synthesized, or manufactured with an anti-inflammatory drug, for example, ibuprofen, diclofenac, aspirin, or naproxen. Additionally or alternatively, a phytochemical composition according to the present disclosure can include or be combined, mixed, synthesized, or manufactured with another substance or composition associated with anti-inflammatory activity, or a substance or composition associated with connective tissue, cartilage, or bone maintenance, support, or regeneration, such as one or more of a glucosamine compound (e.g., glucosamine sulfate or glucosamine hydrochloride), a chondroitin compound (e.g., chondroitin sulfate), methylsulfonylmethane (MSM), or an omega-3 fatty acid. In other embodiments, the phytochemical compositions as disclosed above can be combined, mixed, synthesized, or manufactured with anti-viral drugs, for example oseltamivir (Tamiflu) and zanamivir (Relenza). Additionally or alternatively, particular phytochemical compositions in accordance with the present disclosure can be combined, mixed, synthesized, or manufactured with cholesterol lowering substances or drugs such as statins.

In some embodiments, a combination or mix of a particular phytochemical composition provided by the present disclosure with a particular drug, pharmaceutical composition, phytochemical composition, and/or nutraceutical composition increases the effectiveness, action, and/or safety of the drug, pharmaceutical composition, phytochemical composition, and/or nutraceutical composition, for instance, by way of a synergistic effect. In several embodiments, a combination of a particular phytochemical composition provided by the present disclosure with a particular drug, pharmaceutical composition, phytochemical composition, and/or nutraceutical composition that is associated with anti-viral and/or anti-inflammatory properties increases, enhances, or further facilitates the anti-viral and/or anti-inflammatory effects or actions of the said anti-viral and/or anti-inflammatory drugs, pharmaceutical compositions, phytochemical compositions, and/or nutraceutical compositions, respectively.

Uses of sesamin for the manufacture of phytochemical compositions as disclosed above are also provided by embodiments of the present disclosure. Many embodiments of the present disclosure provide uses of sesamin for the manufacture of phytochemical compositions that include predetermined concentrations of sesamin for therapeutically affecting a pro-inflammatory cytokine condition, for instance facilitating or effectuating decrease in a quantity of pro-inflammatory cytokines, therapeutically affecting an anti-inflammatory cytokine condition, for instance by facilitating or effectuating increase in a quantity of anti-inflammatory cytokines, and/or inhibiting a viral neuraminidase (e.g., influenza A viral neuraminidase) within the living organism.

Several embodiments of the present disclosure provide uses of sesamin for the manufacture of phytochemical compositions that include predetermined concentrations of sesamin for decreasing at least one of pro-inflammatory cytokine gene expression and pro-inflammatory cytokine release by the immune cell. Numerous embodiments of the present disclosure provide uses of sesamin for the manufacture of phytochemical compositions that include predetermined concentrations of sesamin for increasing at least one of anti-inflammatory cytokine gene expression and anti-cytokine release by the immune cell.

Processes and methods for manufacturing phytochemical compositions as disclosed above are also provided by embodiments of the present disclosure. In multiple embodiments, the process for manufacturing the phytochemical composition includes providing a predetermined concentration of sesamin such that the phytochemical composition is able to provide the living organism with a sesamin dosage of at least approximately 5 mg per day. In some embodiments, the process for manufacturing the phytochemical composition includes providing a predetermined concentration of sesamin such that the phytochemical composition is able to provide the living organism with a sesamin dosage of at least approximately 10 mg per day. In selected embodiments, the process for manufacturing the phytochemical composition includes providing a predetermined concentration of sesamin such that the phytochemical composition is able to provide the living organism with a sesamin dosage of at least approximately 20 mg per day.

For increased clarity and understanding of the present disclosure, representative examples of phytochemical compositions, and effects or properties of such phytochemical compositions, are included in the following description. In addition, for the convenience of the reader, general discussions of sesamin, pro-inflammatory cytokines, anti-inflammatory cytokines, viral neuraminidase, and influenza A (H1N1) are also provided in the following disclosure prior to the description of representative examples. It will be understood by a person of ordinary skill in the art that the details presented in the specific examples of the present disclosure are for clarity of illustration and increased understanding, and do not limit the scope of the present disclosure.

### General Discussion of Sesamin

Sesamin is a constituent of sesame oil with an epimer or stereoisomer known as episesamin. The chemical structure of sesamin is:

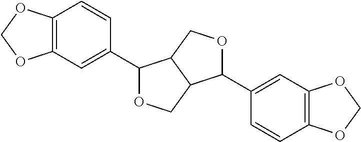 [SMILES: c1cc2c(cc1C1OCC3C(c4ccc5c(c4)OCO5)OCC13)OCO2]

Sesamin is a lignan that is can be extracted from both the bark of Fagara plants and sesame oil. Plant lignans such as sesamin are generally polyphenolic substances that are derived from the phenylalanine amino acid via a dimerization of substituted cinnamic alcohols to a dibenzylbutane skeleton. This dimerization reaction is catalyzed by oxidative enzymes and is typically controlled by dirigent proteins. The term “sesamin” as used in the present disclosure can collectively refer to both sesamin and episesamin.

### General Discussion of Pro-Inflammatory Cytokines

A pro-inflammatory cytokine or a pro-inflammatory mediator is an immuno-regulatory cytokine that favor inflammation. Pro-inflammatory cytokines that are generally responsible for early immune responses include IL-1, IL-6, and TNF-α. IL-1, IL-6, and TNF-α are also considered endogenous pyrogens as they contribute to increasing body temperature. Other examples of pro-inflammatory cytokines or pro-inflammatory mediators include IL-8, IL-11, IL-12, IL-18, GM-CSF, IFN-γ, TGF-β, leukemia inhibitory factors (LIF), oncostatin M (OSM), and a variety of chemokines that attract inflammatory cells.

A pro-inflammatory cytokine generally up-regulates or increases the synthesis of secondary pro-inflammatory mediators and other pro-inflammatory cytokines by immune cells. In addition, pro-inflammatory cytokines can stimulate production of acute phase proteins that mediate inflammation and attract inflammatory cells.

IL-1 is an important pro-inflammatory cytokine. IL-1 is a soluble protein having a mass of approximately 17 kilo-Daltons (kD). IL-1 is produced by a variety of cells, for example macrophages, white blood cells, lymphocytes, monocytes, dendritic cells, and accessory cells that are involved in activation of T-lymphocytes and B-lymphocytes. IL-1 is typically released by such cells during an immune response. IL-1 is generally considered to be a pro-inflammatory cytokine. Pro-inflammatory cytokines generally refer to immunoregulatory cytokines that favor inflammation.

The original members of the IL-1 superfamily are interleukin-1 alpha (IL-1α), interleukin-1 beta (IL-1β), and interleukin-1 receptor antagonist (IL-1RA). Both IL-1α and IL-1β play important roles in the inflammatory response of the body against pathogens or infection. Both IL-1α and IL-1β recognize a same IL-1 receptor and perform similar biological functions. IL-1α is predominantly a cell-associated molecule whereas IL-1β is generally a secreted molecule. The term “IL-1” used in the present disclosure can be taken to include one or both of IL-1α and IL-1β.

The cytokines or interleukins of the IL-1 superfamily (e.g. IL-1α and IL-1β) are generally produced as precursor peptides, which are then processed to become mature proteins (or mature interleukins). For example, each of IL-1α and IL-1β is produced as a precursor peptide. The precursor peptide of each of IL-1α and IL-1β is then processed by specific enzymes to release a smaller active molecule, which is the mature protein. The three dimensional (3D) structure of mature interleukins of the IL-1 superfamily include twelve to fourteen beta (β) strands or beta sheets (which is a secondary structure of proteins) to form barrel-shaped proteins.

IL-1β (also referred to as IL-1B) is encoded by the IL1B gene. Expression of the IL1B gene produces IL-1β precursor peptide. The IL-1β precursor peptide is first cleaved by an enzyme known as caspase 1 or interleukin-1 beta convertase. The product of this reaction is further cleaved by an enzyme known as cytosolic thiol protease to form mature IL-1β. IL-1α (also referred to as IL-1A) is synthesized primarily as a 31 kDa precursor that lacks a signal peptide. The IL-1α precursor is cleaved by a cysteine protease calpain to form mature IL-1α.

IL-1 is produced during immune responses. A common function of IL-1 (e.g. IL-1α and IL-1β) is an increasing of expression of adhesion factors on endothelial cells to enable transmigration of leukocytes (which are immune cells that fight pathogens) to sites of infection. In addition, IL-1 stimulates the hypothalamus thermoregulatory center to cause an increase in body temperature (i.e. a fever). The increased body temperature helps the body's immune system to fight pathogens or infection within the body. In addition, IL-1β is an important mediator of inflammatory response, and is also involved in a range of cellular activities, for example cell proliferation, cell differentiation, and cell apoptosis. Furthermore, IL-1β has also been found to contribute to the sensation of pain during inflammation.

TNF-α is also an important pro-inflammatory cytokine. TNF-α is involved in systemic inflammation and works in tandem with a variety of other cytokines to stimulate the acute phase immune reaction. TNF-α is capable of inducing apoptotic cell death, induce inflammation, as well as inhibit tumorigenesis and viral replication. TNF-α and IL-1 commonly works simultaneously and synergistically in stimulating and sustaining inflammation within the body.

### General Discussion of Anti-Inflammatory Cytokines

Anti-inflammatory cytokines and anti-inflammatory mediators refer generally to immuno-regulatory cytokines that inhibit or counteract various aspects of inflammation. In other words, anti-inflammatory cytokines counteracts various biological effects of pro-inflammatory cytokines and pro-inflammatory mediators. Anti-inflammatory cytokines generally control or mitigate the magnitude of inflammation in vivo. Functions of anti-inflammatory cytokines include inhibiting production of pro-inflammatory cytokines and inhibiting cell activation.

Examples of anti-inflammatory cytokines include IL-2, IL-4, IL-10, and IL-13. IL-2 is a variably glycosylated single protein molecule having as mass of approximately 15.5 kD. IL-2 is generally produced by activated T helper cells (also known as effector T cells) during an immune response. Pathogens (also known as antigens) that invade or are introduced within the body bind to receptors that are found on the surfaces of lymphocytes. Binding of such pathogens or antigens to T cell receptors (TCR) stimulates secretion of IL-2. IL-2 mediates its effects by binding to IL-2 receptor molecules, which are expressed by lymphocytes. The binding of IL-2 to its receptor molecule triggers a signaling cascade, for example Ras/MAPK, JAK/Stat, and PI 3-kinase/Akt signaling modules.

The body produces IL-2 during immune responses. IL-2 has numerous functions including facilitating production of immunoglobulins by B cells. In addition, IL-2 induces differentiation and proliferation of natural killer cells. IL-2 also causes a stimulation of growth, differentiation, and proliferation of antigen-selected cytotoxic T cells via an activation of expression of specific genes. IL-2 is considered to be important for the development of T cell immunologic memory. IL-2 is necessary during T cell development in the thymus for enabling the maturation of regulatory T cells, which are a unique subset of T cells.

### General Discussion of Viral Neuraminidase

A neuraminidase is a glycoside hydrolase enzyme that cleaves the glycosidic linkages of neuraminic acids. A viral neuraminidase is the neuraminidase enzyme present on the surface of a virus, for example an influenza virus.

Enzymatic activity of the viral neuraminidase enables the virus (e.g. the influenza virus) to be released from a host cell. The influenza virus's surface or membrane includes two glycoproteins, namely hemagglutinin and neuraminidase. The presence of hemagglutinin on the surface of the influenza virus inhibits the release of the influenza virus from the host cell. The viral neuraminidase cleaves terminal neuraminic acid residues (also known as sialic acid residues) from glycan structures on the surface of the host cell, thereby enabling the release of the influenza virus from the host cell.

Release of the influenza virus from the host cell causes the spread of the influenza virus to uninfected neighboring cells. In addition, the cleaving of neuraminic acid residues or sialic acid residues from proteins on the surfaces of the influenza viruses prevents an aggregation of the influenza viruses.

The viral neuraminidase is frequently an antigenic determinant for that virus. Accordingly, viral neuraminidase often serves as a target for anti-viral drugs. Drugs (e.g. pharmaceutical compositions) that inhibit the activity of viral neuraminidase have been suggested for use as anti-influenza drugs.

### General Discussion of Influenza A (H1N1)

Influenza, which is commonly referred to as the flu, is an infectious disease caused by RNA viruses of the family Orthomyxoviridae (i.e. influenza viruses). Influenza is typically transmitted through the air or contact with infected body fluids. Common symptoms of influenza include chills, fever, sore throat, muscle pains, headaches, coughing, and general discomfort.

Influenza A (H1N1) is a specific type of influenza that is caused by the influenza A (H1N1) virus. The influenza A (H1N1) virus is a subtype (i.e. a particular strain) of influenza virus A. Influenza A virus strains are typically categorized according to two proteins that are found on the surface of the influenza A virus, namely hemagglutinin (H) and neuraminidase (N). The structure, and number, of each of the two proteins differ between different influenza A virus strains. This difference is due to rapid genetic mutations of the influenza A viral genome. Each influenza virus A strain is assigned a H number and an N number based on the particular structure, form, or number of the H and N proteins on the surface of the influenza A virus.

The influenza A (H1N1) virus is approximately 80-120 nanometers in diameter and is generally spherical in shape. The influenza A (H1N1) virus includes a viral envelope. The viral envelope includes two main types of glycoproteins, which are wrapped about a central core. The central core includes a viral RNA genome and a number of viral proteins that package and protect the viral RNA genome. The viral RNA genome of the influenza A (H1N1) virus includes seven or eight pieces of segmented negative-sense RNA, wherein each piece of RNA includes either one or two genes.

Symptoms of influenza A (H1N1) are similar to that of a regular human flu, and include chills, fever, sore throat, muscle pains, headaches, coughing, and general discomfort. Influenza A (H1N1) has been classified as a global pandemic by the World Health Organization (WHO). The full extent of severity of Influenza A (H1N1) has not been ascertained. However, Influenza A (H1N1) is generally considered to be a significant threat to public health.

The following representative examples describe experiments showing particular effects of phytochemical compositions that include predetermined concentrations of sesamin. It will be understood by a person of ordinary skill in the art that the scope of the present disclosure is not limited to the following examples.

### Example One

Experiments were conducted to evaluate the effect of particular phytochemical compositions provided by the present disclosure on IL-1 gene expression in specified cells found within a body of a living organism. The cells used for the experiments described in example one are lipopolysaccharide (LPS) induced synovial fibroblast (SF) cells. The SF cells were isolated from synovium tissue (also referred to as synovial tissue) using standard cell isolation techniques or methods known to a person of ordinary skill in the art. In addition, LPS is obtained from a commercial source, for example Sigma-Aldrich Co. It is understood by a person having ordinary skill in the art that the SF cells may be substituted with other immune cells found within the body, for example peripheral blood mononuclear cells, lymphocytes, monocytes, and macrophages, which can be isolated using techniques or methods known to a person of ordinary skill in the art. It is also understood by a person having ordinary skill in the art that other inducing substances or chemicals besides LPS, for example viral antigens, may also be used for inducing the SF cells.

Nine cultures of SF cells, i.e. culture 1 to culture 9, were prepared. The contents of the each of cultures 1 to 9 were as follows:
(i) Culture 1: First control culture. Includes a predetermined volume of SF cells.
(ii) Culture 2: Second control culture. Includes the predetermined volume of SF cells induced with 0.2 μg/ml of LPS.
(iii) Culture 3: Third control culture. Includes the predetermined volume of SF cells induced with 0.2 μg/ml of LPS, and 5 μl of dimethyl sulfoxide (DMSO);
(iv) Culture 4: Includes the predetermined volume of SF cells induced with 0.2 μg/ml of LPS, and a phytochemical composition providing 0.025 mM of sesamin;
(v) Culture 5: Includes the predetermined volume of SF cells induced with 0.2 μg/ml of LPS, and a phytochemical composition providing 0.05 mM of sesamin;
(vi) Culture 6: Includes the predetermined volume of SF cells induced with 0.2 μg/ml of LPS, and a phytochemical composition providing 0.10 mM of sesamin;
(vii) Culture 7: Includes the predetermined volume of SF cells induced with 0.2 μg/ml of LPS, and a phytochemical composition providing 0.25 mM of sesamin;
(viii) Culture 8: Includes the predetermined volume of SF cells induced with 0.2 μg/ml of LPS, and a phytochemical composition providing 0.50 mM of sesamin; and
(ix) Culture 9: Includes the predetermined volume of SF cells induced with 0.2 μg/ml of LPS, and a phytochemical composition providing 1.0 mM of sesamin.

In the experiments of example 1, approximately 5.0 ml of SF cells were used for each culture. It will be understood by a person of ordinary skill in the art that alternative volumes of SF cells may also be used. The SF cells were cultured in T-25 flasks. The number or concentration of SF cells used in each of cultures 1 to 9 is approximately equal. For the experiments of example 1, approximately 1×10⁵ (i.e., 100,000) SF cells were present in each culture. It will be understood by a person of ordinary skill in the art that the number or concentration of SF cells used in each of cultures 1 to 9 can be adjusted as required by varying the degree of dilution of the cultures (e.g., by increasing total volume of the culture by adding medium).

Each of cultures 1 to 9 was incubated at the same physical conditions (e.g., same temperate, oxygen concentration, and amount of agitation) for an equal length of time, for instance 24 hours.

The amount or level of IL-1 gene expression in the SF cells was measured or determined using methods or techniques known to a person having ordinary skill in the relevant art. In example 1, Real-Time PCR (RT-PCR) was used for quantifying the amount of IL-1 gene expression in the SF cells. IL-1beta or IL-1β specific primers were used during RT-PCR, thereby facilitating the quantification of IL-1β gene expression in the SF cells. However, it will be understood by a person having ordinary skill in the relevant art that IL-1β gene expression directly corresponds to or mirrors IL-1βα gene expression, and therefore the overall IL-1 gene expression in the SF cells.

### Results

The IL-1 gene expression relative to glyceraldehydes-3-phosphate dehydrogenase (GAPDH) gene expression of SF cells in each of cultures 1 to 9 was measured using RT-PCR. Results show that the IL-1 gene expression relative to GAPDH was lower in SF cells of cultures that were incubated with phytochemical compositions that include predetermined concentrations of sesamin. The IL-1 gene expression relative to GAPDH decreased by approximately 20% and approximately 65% in cultures 4 to 9, i.e. in cultures that were incubated with phytochemical compositions that include predetermined concentrations of sesamin, as compared to any one of control cultures 1 to 3. The maximal decrease in gene expression is seen with culture 9, which comprises the phytochemical composition having sesamin concentration of 1.0 mM. The IL-1 gene expression relative to GAPDH decreased by approximately 65% in culture 9 as compared to control culture 2. The minimal decrease in gene expression is seen with culture 4, which comprises the phytochemical composition having sesamin concentration of 0.025 mM. The IL-1 gene expression relative to GAPDH decreased by approximately 20% in culture 4 as compared to control culture 3. Results indicate that cultures incubated with phytochemical compositions having higher concentrations of sesamin produce a correspondingly greater decrease in IL-1 gene expression relative to GAPDH in SF cells.

### Conclusion

The experiment of example one shows that phytochemical compositions that include sesamin are able to reduce IL-1 gene expression in cells found within the body (e.g., synovial fibroblast cells). More specifically, phytochemical compositions that include sesamin are able to reduce IL-1 gene expression in specific cells of the body by between approximately 20% and approximately 65%. In addition, phytochemical compositions with higher concentrations of sesamin are able to produce a greater decrease in IL-1 gene expression in cells of the body. Although the cells used for the experiment described in example one are SF cells, a person of ordinary skill in the art will understand that the effects of the phytochemical compositions that include sesamin can be replicated or demonstrated with other immune cells found within the body.

### Example Two

Experiments were conducted to evaluate the effect of particular phytochemical compositions provided by the present disclosure on quantity of IL-1 released by specified cells found within a body of a living organism. Cells used for the experiment described in this example are LPS induced peripheral blood mononuclear cells (PBMCs). It is understood by a person of ordinary skill in the art that the PBMCs may be substituted for other immune cells found within the body. It is also understood by a person having ordinary skill in the art that other inducing substances or chemicals besides LPS, for example viral antigens, may also be used for inducing the PBMCs.

To conduct the experiments of example two, PBMCs were isolated and prepared using isolation techniques or methods known to a person of ordinary skill in the art. An exemplary method or technique is the differential density gradient centrifugation technique, the steps or process steps of which are known to a person of ordinary skill in the art. In addition, LPS can be obtained from a commercial source, for example Sigma-Aldrich Co.

The PBMCs are cultured in 10% fetal calf serum in RPMI1640 medium. Seven cultures of PBMCs, i.e. culture 1 to culture 7, were prepared. The contents of each of cultures 1 to 7 are as follows:
(i) Culture 1: First control culture. Includes a predetermined volume of PBMCs (1×10⁵ PBMCs/culture);
(ii) Culture 2: Second control culture. Includes the predetermined volume of PBMCs (1×10⁵ PBMCs/culture) and LPS at a concentration of 20 ng/ml;
(iii) Culture 3: Includes the predetermined volume of the specified concentration of PBMCs, LPS at a concentration of 20 ng/ml, and a volume of phytochemical composition providing 0.1 uM of sesamin;
(iv) Culture 4: Includes the predetermined volume of PBMCs (1×10⁵ PBMCs/culture), LPS at a concentration of 20 ng/ml, and a volume of phytochemical composition providing 0.5 uM of sesamin;
(v) Culture 5: Includes the predetermined volume of PBMCs (1×10⁵ PBMCs/culture), LPS at a concentration of 20 ng/ml, and a volume of phytochemical composition providing 1.0 uM of sesamin;
(vi) Culture 6: Includes the predetermined volume of PBMCs (1×10⁵ PBMCs/culture), LPS at a concentration of 20 ng/ml, and a volume of phytochemical composition providing 5.0 uM of sesamin; and
(vii) Culture 7: Includes the predetermined volume of PBMCs (1×10⁵ PBMCs/culture), LPS at a concentration of 20 ng/ml, and a volume of phytochemical composition providing 10 uM of sesamin.

In the experiments of example 2, each of the cultures 1 to 7 is made up to a final volume of approximately 100 μl in a well of a standard 96 well plate. It will be understood by a person of ordinary skill in the art that alternative volumes may also be used. The number or concentration of PBMCs used for each culture is approximately equal. For instance, the number of PBMCs used for each culture is approximately 1×10⁵ (i.e., 100,000). It will be understood by a person of ordinary skill in the art that the number or concentration of PBMCs used for each of cultures 1 to 7 can be adjusted as required by varying the degree of dilution of the cultures (e.g., by increasing total volume of the culture by adding medium).

Each culture was incubated at the same physical conditions for an equal length of time. More specifically, each culture was incubated at a temperature of 37° C. with 5% of carbon dioxide for 24 hours.

After a predetermined time period, quantity of IL-1 in each of cultures 1 to 7 was quantified using the ELISA technique (e.g., Biotrak Easy ELISA Aersham). The quantity of IL-1 detected by the ELISA technique in each of cultures 1 to 7 reflects the quantity of IL-1 released by the PBMCs of that culture.

### Results

Results showed that quantity or level of IL-1 (pg/ml) was lower in cultures that were incubated with phytochemical compositions that included sesamin (i.e., in cultures 3 to 7) as compared to cultures that did not include any concentration of sesamin (i.e., in cultures 1 and 2). More specifically, the quantity of IL-1 (pg/ml) decreased by between approximately 5% and approximately 35% in cultures that were incubated with phytochemical compositions that include sesamin (i.e., in cultures 3 to 7). The minimum decrease was observed with cultures 3 and 4, which showed an approximately 5% decrease in quantity of IL-1 as compared to control culture 1. The maximum decrease was observed with culture 7, which showed an approximately 35% decrease in quantity of IL-1 as compared to control culture 2. Results indicate that the quantity of IL-1 decreased by a greater amount in cultures that were incubated with phytochemical compositions having higher concentrations of sesamin.

### Conclusion

The experiments of example two indicate that phytochemical compositions that include sesamin are able to reduce the amount or quantity of IL-1 released by specified cells (e.g. PMBCs) found within a body of a living organism. This reduction of quantity of IL-1 released is between approximately 5% and approximately 35%. In addition, phytochemical compositions having higher concentrations of sesamin are able to facilitate or effectuate a greater decrease in the amount or quantity of IL-1 released by specified cells of the body. Although the cells used for the experiments described in example two are PMBCs, a person having ordinary skill in the art will understand that the effects of the phytochemical compositions having sesamin can be replicated or demonstrated with other immune cells found within the body.

### Example Three

Experiments were conducted to evaluate the effect of particular phytochemical compositions provided by the present disclosure on IL-2 gene expression in specified cells found within a body of a living organism. Cells used for the experiment described in this example are LPS induced peripheral blood mononuclear cells (PBMCs). It is understood by a person having ordinary skill in the art that the PBMCs may be substituted for other immune cells found within the body. It is also understood by a person having ordinary skill in the art that other inducing substances or chemicals besides LPS, for example viral antigens, may also be used for inducing the PBMCs.

Preparation of PBMCs is done by standard isolation techniques or methods known to a person of ordinary skill in the art as with the experiments of example two. In addition, LPS can be obtained from a commercial source, for example Sigma-Aldrich Co. The PBMCs are cultured in 10% fetal calf serum in RPMI1640 medium. Five cultures of PBMCs, i.e. culture 1 to culture 5, were prepared. The contents of each of cultures 1 to 5 were as follows:
(i) Culture 1: Control culture. Includes a predetermined volume of PBMCs (1×10⁵ PBMCs/culture);
(ii) Culture 2: Includes the predetermined volume of PBMCs (1×10⁵ PBMCs/culture), LPS at a concentration of 20 ng/ml, and a volume of phytochemical composition providing 0.1 ug/ml of sesamin;
(iii) Culture 3: Includes the predetermined volume of PBMCs (1×10⁵ PBMCs/culture), LPS at a concentration of 20 ng/ml, and a volume of phytochemical composition providing 0.5 ug/ml of sesamin;
(iv) Culture 4: Includes the predetermined volume of PBMCs (1×10⁵ PBMCs/culture), LPS at a concentration of 20 ng/ml, and a volume of phytochemical composition providing 1.0 ug/ml of sesamin; and
(v) Culture 5: Includes the predetermined volume of PBMCs (1×10⁵ PBMCs/culture), LPS at a concentration of 20 ng/ml, and a volume of phytochemical composition providing 5.0 ug/ml of sesamin.

In the experiments of example 3, each of the cultures 1 to 5 is made up to a final volume of approximately 100 μl in standard 96 well plates. It will be understood by a person of ordinary skill in the art that alternative volumes may also be used. The number or concentration of PBMCs used for each culture is approximately equal. For instance, the number of PBMCs used for each culture is approximately 1×10⁵ (i.e., 100,000). It will be understood by a person of ordinary skill in the art that the number or concentration of PBMCs used for each of cultures 1 to 5 can be adjusted as required by varying the degree of dilution of the cultures (e.g., by increasing total volume of the culture by adding medium).

Each of cultures 1 to 5 was incubated at the same physical conditions for an equal length of time. More specifically, each culture was incubated at a temperature of 37° C. with 5% of carbon dioxide for 24 hours.

The amount or level of IL-2 gene expression in the PBMCs was measured or determined using methods or techniques known to a person having ordinary skill in the relevant art. In example three, Real-Time PCR (RT-PCR) was used for quantifying the amount of IL-2 gene expression in the PBMCs.

### Results

Results showed that the level of IL-2 gene expression relative to GAPDH increases in PBMCs of cultures that were incubated with phytochemical compositions having predetermined concentrations of sesamin (i.e. cultures 2 to 5). More specifically, the results showed that phytochemical compositions including predetermined concentrations of sesamin produces an increase in IL-2 gene expression relative GAPDH of between approximately 40% and approximately 220%. The increase in IL-2 gene expression relative GAPDH is greatest, more specifically approximately 220%, in PBMCs that were incubated with the phytochemical composition having 1.0 ug/ml of sesamin (i.e. in culture 4). Results indicate that the increase in IL-2 gene expression in PBMCs increases when the PBMCs are incubated with phytochemical compositions of increasing sesamin concentration, more specifically from 0.1 ug/ml to 1.0 ug/ml. Results suggest that maximal increase in IL-2 gene expression is achieved with phytochemical compositions of sesamin concentrations less than 5.0 ug/ml.

### Conclusion

The experiments of example three show that phytochemical compositions that include sesamin increase IL-2 gene expression in specific cells found within the body (e.g. PBMCs). More specifically, phytochemical compositions that include sesamin increase IL-2 gene expression in specific cells of the body by between approximately 40% and approximately 220%. Phytochemical compositions having increasing concentrations of sesamin from approximately 0.1 ug/ml to approximately 1.0 ug/ml produce the increasing increase in IL-2 gene expression in the cells of the body. However, maximal increase in IL-2 gene expression in the cells of the body is achieved with phytochemical compositions having sesamin concentrations of less than approximately 5.0 ug/ml. Although the cells used for the experiment described in example three are PMBCs, a person having ordinary skill in the art will understand that the effects of the phytochemical compositions including sesamin can be replicated or demonstrated with other immune cells found within the body.

### Example Four

A computer graphic simulation and demonstration showing an inhibitory effect of sesamin against viral neuraminidase is also provided by the present disclosure. More specifically, the computer graphic simulation provided by example four shows the inhibitory effect of sesamin against the neuraminidase of the influenza A (H1N1) virus. Sesamin binds to specific sites of the neuraminidase of the influenza A (H1N1) virus via at least one functional group of the sesamin molecule. Computer simulation showed that a binding energy of approximately −29.1 kcal/mol was associated with the specific binding between the at least one functional group of sesamin and the neuraminidase of the influenza A (H1N1) virus. The binding energy of −29.1 kcal/mol indicates that the binding of sesamin with the neuraminidase of the influenza A (H1N1) virus facilitates a decrease or an inactivation of action or activity of the influenza A (H1N1) virus.

### Conclusion

The computer graphic simulation and demonstration provided by example four shows that sesamin includes at least one functional group that is capable of binding to viral neuraminidase (e.g., neuraminidase of the influenza A (H1N1) virus). The binding energy of the bond formed between the sesamin molecule and the viral neuraminidase indicates that the binding of sesamin to the viral neuraminidase results in a more stable sesamin-viral neuraminidase compound relative to viral neuraminidase when considered alone. Accordingly, the binding energy indicates that the binding of the sesamin molecule with the viral neuraminidase can inactivate or reduce the action or activity of the viral neuraminidase. The sesamin facilitated inactivation or reduction in action or activity of the viral neuraminidase confers upon sesamin, and phytochemical compositions including sesamin, an anti-viral (e.g. anti-influenza A (H1N1)) or viral modulatory property or use.

### Example Five

Experiments were conducted to evaluate the anti-inflammatory effects of combinations of particular phytochemical compositions provided by the present disclosure with known substances, compositions, or drugs that are associated with an anti-inflammatory effect and/or an anti-degenerative or tissue integrity preservation effect in connective tissue. In the context of the present disclosure, the term “connective tissue” encompasses connective tissue proper (e.g., dense or fibrous connective tissue), as well as specialized connective tissue such as cartilage or bone. More specifically, experiments were conducted to evaluate the anti-inflammatory, anti-degenerative, and/or structural preservation effects of a combination of particular phytochemical compositions provided by the present disclosure with glucosamine sulfate. In addition, the experiments compared the anti-inflammatory/anti-degenerative effects of the combination of particular sesamin or sesamin-based phytochemical compositions provided by the present disclosure plus glucosamine sulfate, versus the anti-inflammatory/anti-degenerative effects of glucosamine sulfate alone.

Hyaluronic acid (HA) is an important component of the extracellular matrix (ECM), as well as a major component of synovial fluid. HA is present in articular cartilage, and contributes to cartilage resilience. It is known that IL-1 stimulates the synthesis and activity of matrix metalloproteinases and other enzymes that are associated with cartilage degradation as well as rheumatoid arthritis and osteoarthritis. A set of experimental cartilage tissue cultures were prepared using cartilage tissue samples containing HA within their ECMs. As further detailed below, the experimental cartilage tissue cultures selectively incorporated glucosamine sulfate alone; sesamin alone; or a combination of glucosamine sulfate and sesamin. Additionally, a control cartilage tissue culture remained unexposed to either of sesamin or glucosamine sulfate.

ECM degradation was induced in the experimental and control cartilage tissue cultures by exposing the cultures to IL-1. A measure or estimate of a cartilage tissue anti-degenerative effect was defined in accordance with an amount of HA that an experimental cartilage tissue culture released into the tissue culture medium relative to a reference or baseline amount of HA that was released into the tissue culture medium by the control cartilage tissue culture. More particularly, a percentage reduction in the amount of HA released into the tissue culture medium by a given experimental cartilage tissue culture relative to the amount of HA released into the tissue culture medium by the control cartilage tissue culture, indicated below as “Percentage (%) HA Reduction”, conveys or indicates the magnitude of an anti-degenerative or structural preservation effect provided by the experimental cartilage tissue culture under consideration. A corresponding measure or estimate of percentage (%) HA retention by cartilage tissue can also be defined based upon the experimental results. The amount or level of HA present in a given tissue culture medium was detected using ELISA, in a manner that can be understood by one of ordinary skill in the art.

### Results

TABLE 1
Effect of different approximate concentrations of glucosamine sulfate
on approximate percentage (%) HA reduction.

| Glucosamine Sulfate | | Percentage (%) HA |
|---|---|---|
| Concentration (mM) | Concentration (mg/ml) | Reduction |
| 20 | 9 | 3 |
| 40 | 18 | 28 |
| 80 | 36 | 43 |

TABLE 2
Effect of different approximate concentrations of sesamin on
approximate percentage (%) HA reduction.

| Sesamin | | Percentage (%) HA |
|---|---|---|
| Concentration (uM) | Concentration (ug/ml) | Reduction |
| 0.25 | 88.6 | 0 |
| 0.50 | 177.2 | 4 |
| 1.00 | 354.4 | 56 |

TABLE 3
Effect of different approximate concentrations
of a combination of glucosamine sulfate and
sesamin on approximate percentage (%) HA reduction.

Glucosamine Sulfate/Sesamin

| Concentration | Concentration | Percentage (%) HA |
|---|---|---|
| (mM)/(uM) | (mg/ml): (ug/ml) | Reduction |
| 2.5/0.0312 | 1.13/11.1 | 17 |
| 5/0.0625 | 2.25/22.2 | 41 |
| 10/0.125 | 4.5/44.3 | 57 |
| 20/0.25 | 9/88.6 | 64 |
| 40/0.5 | 18/177.2 | 77 |
| 80/1.0 | 36/354.4 | 109 |

### Conclusion

The foregoing results show that particular phytochemical compositions provided by the present disclosure (i.e., phytochemical compositions including predetermined concentrations of sesamin) facilitate or provide an anti-inflammatory, anti-degeneration, or ECM preservation effect upon cartilage tissue. Additionally, combinations of particular sesamin or sesamin-based phytochemical compositions with glucosamine sulfate provide an enhanced or synergistic anti-inflammatory, anti-degeneration, or ECM preservation effect upon cartilage tissue compared to glucosamine sulfate alone.

Additionally, the magnitude of the synergistic effect was surprisingly or unexpectedly strong. For instance, while glucosamine sulfate alone at an approximate concentration of 20 mM (or about 9 mg/ml) gave rise to a percentage reduction of HA released into the tissue culture medium of approximately 3% relative to the amount of HA released into the tissue culture medium by the control culture; and sesamin alone at an approximate concentration of 0.25 uM (or about 88.6 ug/ml) gave rise to a percentage reduction of HA released into the tissue culture medium of approximately 0% relative to the control culture release, the combination of glucosamine sulfate and sesamin at these concentrations (i.e., about 20 mM glucosamine sulfate plus about 0.25 uM sesamin, or about 9 mg/ml glucosamine sulfate and about 88.6 ug/ml sesamin) gave rise to a percentage reduction of HA released into the tissue culture medium of approximately 64% relative to the amount of HA released into the tissue culture medium by the control culture. Thus, the actual result indicates a synergistic effect that is much greater than a simple independent additive effect that would correspond to a percentage (%) HA reduction of approximately 3%+0% relative to the control culture results. Furthermore, the combination of glucosamine sulfate and sesamin at these concentrations (i.e., about 20 mM glucosamine sulfate plus about 0.25 uM sesamin, or about 9 mg/ml glucosamine sulfate and about 88.6 ug/ml sesamin) gave rise to an approximately 2000% increase in the percentage reduction of HA released into the tissue culture medium as compared to that with approximately 20 mM or 9 mg/ml of glucosamine sulfate alone. Thus, the combination of a glucosamine compound (e.g., glucosamine sulfate) and sesamin is expected to facilitate or give rise to a substantially or very substantially greater anti-inflammatory and/or connective tissue anti-degenerative or ECM preservation effect than that which would be provided by the glucosamine compound alone (e.g., by at least approximately 50%, at least approximately 100%, or greater than 100%).

Moreover, while glucosamine sulfate alone at an approximate concentration of 40 mM or 18 mg/ml gave rise to a percentage reduction of HA released into the tissue culture medium of approximately 28%; and sesamin alone at an approximate concentration of 0.50 uM or 177.2 ug/ml gave rise to a percentage reduction of HA released into the tissue culture medium of approximately 4%, the combination of glucosamine sulfate and sesamin at these concentrations (i.e., about 40 mM glucosamine sulfate plus about 0.50 uM sesamin) gave rise to a percentage reduction of HA released into the tissue culture medium of approximately 77%. Additionally, the combination of glucosamine sulfate and sesamin at these concentrations (i.e., about 40 mM glucosamine sulfate plus about 0.50 uM sesamin, or about 18 mg/ml glucosamine sulfate and about 177.2 ug/ml sesamin) gave rise to at least an approximately 175% increase in the percentage reduction of HA released into the tissue culture medium as compared to either of glucosamine sulfate (i.e., about 40 mM or about 18 mg/ml) or sesamin (i.e., about 0.5 uM or about 177.2 ug/ml) when taken alone.

In addition, while glucosamine sulfate alone at an approximate concentration of 80 mM or 36 mg/ml gave rise to a percentage reduction of HA released into the tissue culture medium of approximately 43%; and sesamin alone at an approximate concentration of 1.0 uM or about 354.4 ug/ml gave rise to a percentage reduction of HA released into the tissue culture medium of approximately 43%, the combination of glucosamine sulfate and sesamin at these concentrations (i.e., about 80 mM glucosamine sulfate plus about 1.0 uM sesamin) gave rise to a percentage reduction of HA released into the tissue culture medium of approximately 109%. Correspondingly, the combination of glucosamine sulfate and sesamin at these concentrations (i.e., about 80 mM or about 36 mg/ml glucosamine sulfate plus about 1.0 uM or about 354.4 ug/ml sesamin) gave rise to at least an approximately 150% increase in the percentage reduction of HA released into the tissue culture medium as compared to either of glucosamine sulfate (i.e., about 80 mM or 36 mg/about ml) or sesamin (i.e., about 1.0 uM or about 354.4 ug/ml) when taken alone.

In view of the foregoing, neither glucosamine sulfate by itself at a concentration at or below approximately 10 mM or 4.5 mg/ml, nor sesamin by itself at a concentration at or below approximately 0.125 uM or 44.3 ug/ml would be expected to result in a noticeable or significant percentage reduction in HA released from an ECM. However, the synergistic combination of glucosamine sulfate with sesamin at a glucosamine sulfate: sesamin concentration of approximately 10 mM: 0.125 uM, or approximately 4.5 mg/ml: 44.3 ug/ml, gave rise to approximately a 57% percent reduction in HA released by the ECM into the tissue culture medium compared with the control culture. In several embodiments, a composition in accordance with the present disclosure directed to facilitating an anti-inflammatory, anti-degenerative, or structural integrity maintenance effect (e.g., an ECM preservation effect) upon connective tissue, cartilage, or bone can include or provide (e.g., in association with a single daily dose, or series of doses that contribute to a daily dose) a glucosamine sulfate concentration of at least about 2.5 mM or about 1.13 mg/ml, plus a sesamin concentration of at least about 0.0312 uM or about 11.1 ug/ml. In some embodiments, such a composition can include or supply at least about 5.0 mM or 2.25 mg/ml of glucosamine sulfate (e.g., at least approximately 10, 20, 40, or 80 mM or respectively 4.5, 9.0, 18.0, or 36 mg/ml of glucosamine sulfate); and about 0.0625 uM or 22.2 ug/ml of sesamin (e.g., at least approximately 0.125, 0.25, 0.50 or 1.0 uM or respectively 44.3, 88.6, 177.2, or 354.4 ug/ml of sesamin).

In accordance with certain embodiments of the present disclosure, a phytochemical composition that includes or is derived from sesamin and one or more of a glucosamine compound, a chondroitin compound, MSM, and/or another compound that can directly or indirectly contribute to ECM structural integrity can provide an anti-inflammatory and/or a structural preservation effect to one or more of connective tissue, cartilage, or bone.

Additionally, combinations of particular phytochemical compositions provided by the present disclosure (i.e., phytochemical compositions including predetermined concentrations of sesamin) and existing anti-inflammatory drugs or pharmaceutical compositions can correspondingly provide an enhanced anti-inflammatory effect or use as compared to exiting anti-inflammatory drugs or pharmaceutical compositions when taken alone. Particular phytochemical compositions provided by the present disclosure facilitate or produce synergistic effects when taken in combination with existing anti-inflammatory drugs or pharmaceutical compositions.

### Example Six

Experiments were conducted to evaluate the effects of particular phytochemical compositions provided by the present disclosure on quantity of IL-1 released by virus-infected cells. Virus-infected cells used for the experiments described in this example are influenza A (H1N1) virus-infected cells. More specifically, the virus-infected cells used for the experiments described in this example are influenza A (H1N1) virus-infected egg cells. The influenza A (H1N1) virus can be isolated from patients infected with the influenza A (H1N1) using standard techniques known to a person of ordinary skill in the art. In addition, isolation of cells (e.g., egg cells), and the infection of cells (e.g., isolated egg cells) can be performed using standard techniques or method known to a person of ordinary skill in the art. It is understood by a person having ordinary skill in the art that the influenza A (H1N1) virus-infected egg cells may be substituted for other viral-infected cells, or other cells found within the body, for instance, cells that that are involved in inflammatory responses within the body.

Six cultures of cells, i.e., culture 1 to culture 6, were prepared. The contents of each of cultures 1 to culture 6 are as follows:
(i) Culture 1: Control culture 1. Includes a predetermined volume of a sample of isolated egg cells that has been diluted in a ratio of 1:50;
(ii) Culture 2: Control culture 2. Includes the predetermined volume of the sample of isolated egg cells similar to that of culture 1 that have been infected with the influenza A (H1N1) virus diluted at a ratio of 1:7812500;
(iii) Culture 3: Control culture 3. Includes the predetermined volume of the sample of isolated egg cells similar to that of culture 1 and a volume of DMSO that has been infected with the influenza A (H1N1) virus diluted at a ratio of 1:7812500;
(iv) Culture 4: Includes the predetermined volume of the sample of isolated egg cells that have been infected with the influenza A (H1N1) virus similar to that of culture 2, and a volume of phytochemical composition providing sesamin at a concentration of 0.5 ug/ml;
(v) Culture 5: Includes the predetermined volume of the sample of isolated egg cells that have been infected with the influenza A (H1N1) virus similar to that of culture 2, and a volume of phytochemical composition providing sesamin at a concentration of 1.0 ug/ml; and
(vi) Culture 6: Includes the predetermined volume of the sample of isolated egg cells that have been infected with the influenza A (H1N1) virus similar to that of culture 2, and a volume of phytochemical composition providing sesamin at a concentration of 10.0 ug/ml.

In the experiments of example six, each of cultures 1 to 6 is made up to a final volume of approximately 200 μl in a well of a standard 96 well plate. It will be understood by a person of ordinary skill in the art that an alternative final volume (e.g., 100 μl) may also be used. The number or concentration of isolated egg cells used for each culture is approximately equal. For instance, the number of isolated egg cells used for each culture is approximately 2×10⁵ (i.e., 200,000). It will be understood by a person of ordinary skill in the art that the number or concentration of isolated egg cells used for each of cultures 1 to 6 can be adjusted as required by varying the degree of dilution of the cultures (e.g., by increasing total volume of the culture by adding medium).

Each of cultures 1 to 6 was incubated at the same physical conditions for an equal length of time. More specifically, each culture was incubated at a temperature of 37° C. with 5% of carbon dioxide for 24 hours. The amount of IL-1 released from the isolated egg cells of each of cultures 1 to 6 was measured or determined using methods or techniques known to a person having ordinary skill in the relevant art.

### Results

Results show that the quantity of IL-1 released from cells that are infected with the influenza A (H1N1) virus (i.e., cells of control cultures 2 and 3) is higher than the quantity of IL-1 released from cells that are not infected with the influenza A (H1N1) virus (i.e., cells of control culture 1). More specifically, approximately between 28 pg/ml and 31 pg/ml of IL-1 was released by influenza A (H1N1) virus-infected cells (i.e., cells of control cultures 2 and 3) as compared to approximately 19 pg/ml of IL-1 that was released by cells that were not viral-infected (i.e., cells of control culture 1). In other words, the quantity of IL-1 released by influenza A (H1N1) virus-infected cells (i.e., cells of control cultures 2 and 3) increases by between approximately 45% and 65% as compared to the quantity of IL-1 released by cells that were not viral infected (i.e., cells of control culture 1). Such an increase in quantity of IL-1 released corresponds to a significant immune response in the body of a living organism.

Results show that the quantity of IL-1 released from the influenza A (H1N1) virus-infected cells is lower in cultures that further include phytochemical compositions that have predetermined concentrations of sesamin (i.e., 0.5 ug/ml, 1.0 ug/ml, and 10.0 ug/ml).

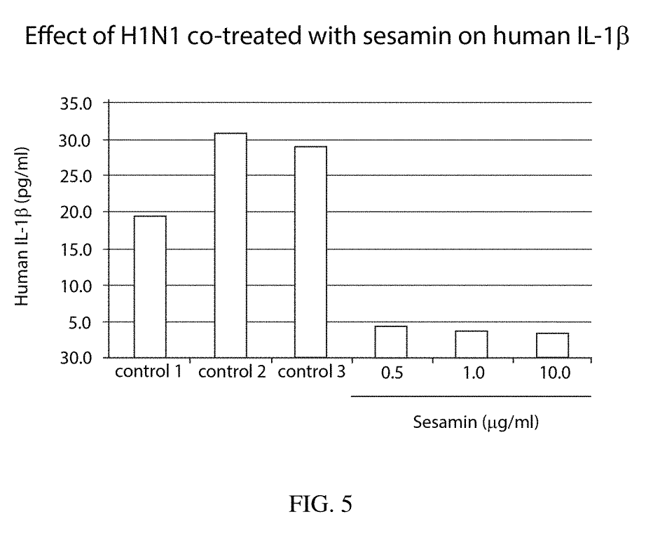

As shown in FIG. 5, the quantity of IL-1 released by influenza A (H1N1) virus-infected cells incubated with phytochemical compositions including 0.5 ug/ml of sesamin (i.e., in culture 4) was approximately 4 pg/ml. This represents between approximately 25 pg/ml and 27 pg/ml decrease in the quantity of IL-1 released by influenza A (H1N1) virus-infected cells when said cells are incubated with phytochemical compositions including 0.5 ug/ml of sesamin as compared to when said cells were not incubated with any amount of sesamin (i.e., in control cultures 2 and 3).

As also shown in FIG. 5, the quantity of IL-1 released by influenza A (H1N1) virus-infected cells incubated with phytochemical compositions including 1.0 ug/ml of sesamin (i.e., in culture 5) was approximately 3.5 pg/ml. This represents between approximately 25.5 pg/ml and 27.5 pg/ml decrease in the quantity of IL-1 released by influenza A (H1N1) virus-infected cells when said cells were incubated with phytochemical compositions including 1.0 ug/ml of sesamin as compared to when said cells were not incubated with any amount of sesamin (i.e., in control cultures 2 and 3).

Furthermore, FIG. 5 also shows that the quantity of IL-1 released by influenza A (H1N1) virus-infected cells incubated with phytochemical compositions including 10.0 ug/ml of sesamin (i.e., in culture 6) was approximately 3 pg/ml. This represents between approximately 26 pg/ml and 28 pg/ml decrease in the quantity of IL-1 released by influenza A (H1N1) virus-infected cells when said cells were incubated with phytochemical compositions including 10.0 ug/ml of sesamin as compared to when said cells were not incubated with any amount of sesamin (i.e., in control culture 2).

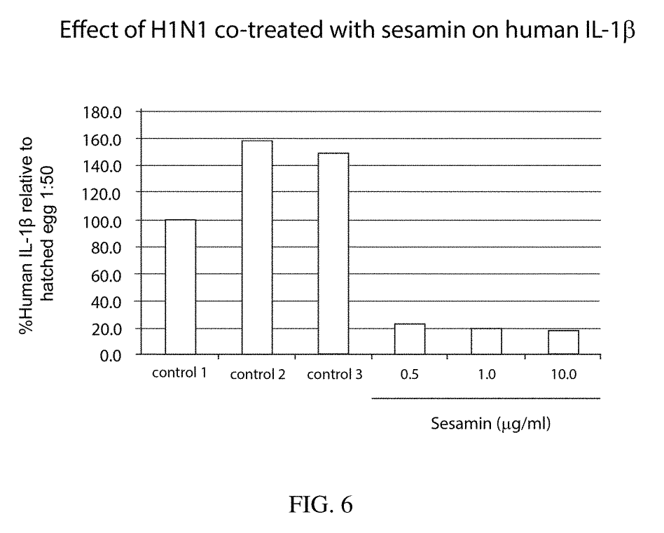

As shown in FIG. 6, the percentage of quantity of human IL-1 relative the quantity of isolated egg cells (diluted in a ratio of 1:50) was determined for each of cultures 1 to 6.

Results show that the percentage of quantity of human IL-1 relative the quantity of isolated egg cells (diluted in a ratio of 1:50) increased when the isolated egg cells were infected with the influenza A (H1N1) virus diluted at a ratio of 1:7812500. As shown in FIG. 6, the percentage of quantity of human IL-1 relative the quantity of isolated egg cells (diluted in a ratio of 1:50) increased from 100% to between approximately 145% and 160% when the isolated egg cells were infected with the influenza A (H1N1) virus diluted at a ratio of 1:7812500 (i.e., in cultures 2 and 3) as compared to when said cells were not infected with the influenza A (H1N1) virus diluted at a ratio of 1:7812500 (i.e., in culture 1).

Results also show that the percentage of quantity of human IL-1 relative the quantity of isolated egg cells (diluted in a ratio of 1:50) decreased when the cells that were infected with the influenza A (H1N1) virus diluted at a ratio of 1:7812500 were incubated with the phytochemical compositions including predetermined concentrations of sesamin (e.g., 0.5 ug/ml, 1.0 ug/ml, and 10.0 ug/ml) as compared to when said cells were not incubated with any concentration of sesamin.

As shown in FIG. 6, the percentage of quantity of human IL-1 released relative to quantity of isolated egg cells diluted in a ration of 1:50) was approximately 22% when isolated eggs cells that were infected with the influenza A (H1N1) virus diluted at a ratio of 1:7812500 were incubated with the phytochemical composition including 0.5 ug/ml of sesamin. This represents between an approximately 123% and an approximately 138% decrease in the quantity of human IL-1 released relative to quantity of isolated egg cells diluted in a ration of 1:50) when the isolated eggs cells that were infected with the influenza A (H1N1) virus diluted at a ratio of 1:7812500 were incubated with the phytochemical composition including 0.5 ug/ml of sesamin (i.e., in culture 4) as compared to when the said cells were not incubated with any concentration of sesamin (i.e., in cultures 2 and 3).

As also shown in FIG. 6, the percentage of quantity of human IL-1 released relative to quantity of isolated egg cells diluted in a ration of 1:50) was approximately 18% when isolated eggs cells that were infected with the influenza A (H1N1) virus diluted at a ratio of 1:7812500 were incubated with the phytochemical composition including 1.0 ug/ml of sesamin. This represents between an approximately 127% and an approximately 142% decrease in the quantity of human IL-1 released relative to quantity of isolated egg cells diluted in a ration of 1:50) when the isolated eggs cells that were infected with the influenza A (H1N1) virus diluted at a ratio of 1:7812500 were incubated with the phytochemical composition including 1.0 ug/ml of sesamin (i.e., in culture 5) as compared to when the said cells were not incubated with any concentration of sesamin (i.e., in cultures 2 and 3).

As further shown in FIG. 6, the percentage of quantity of human IL-1 released relative to quantity of isolated egg cells diluted in a ration of 1:50) was approximately 16% when isolated eggs cells that were infected with the influenza A (H1N1) virus diluted at a ratio of 1:7812500 were incubated with the phytochemical composition including 10.0 ug/ml of sesamin. This represents between an approximately 129% and an approximately 144% decrease in the quantity of human IL-1 released relative to quantity of isolated egg cells diluted in a ration of 1:50) when the isolated eggs cells that were infected with the influenza A (H1N1) virus diluted at a ratio of 1:7812500 were incubated with the phytochemical composition including 10.0 ug/ml of sesamin (i.e., in culture 6) as compared to when the said cells were not incubated with any concentration of sesamin (i.e., in cultures 2 and 3).

### Conclusion

The experiments of example six show that influenza A (H1N1) virus stimulates an increased release of IL-1 from isolated egg cells. In addition, the experiments of example six show that the incubation of isolated egg cells infected with influenza A (H1N1) with phytochemical compositions including approximately 0.5 ug/ml and approximately 10.0 ug/ml of sesamin decreases the quantity of IL-1 released from the said cells. More specifically, the incubation of isolated egg cells infected with influenza A (H1N1) with phytochemical compositions including approximately 0.5 ug/ml and approximately 10.0 ug/ml of sesamin results in between an approximately 25 pg/ml and an approximately 28 pg/ml decrease in IL-1 released from the said cells as compared to when the said cells were not incubated with any concentration of sesamin. In addition, the incubation of isolated egg cells infected with influenza A (H1N1) with phytochemical compositions including approximately 0.5 ug/ml and approximately 10.0 ug/ml of sesamin results in a decrease of the percentage of quantity of human IL-1 relative to isolated egg cells (diluted in a ratio of 1:50) by between approximately 123% and approximately 144%. Although the cells used for the experiment described in example six are isolated egg cells infected with influenza A (H1N1), a person having ordinary skill in the art will understand that the effects of the phytochemical compositions including sesamin can be replicated or demonstrated with other viral infected cells or other cells that are involved in inflammation within the body.

Embodiments of the present disclosure provide compositions (e.g., phytochemical compositions) that include predetermined concentrations of sesamin for at least one, and in some embodiments two or more, of reducing quantity of a pro-inflammatory cytokine, increasing quantity of an anti-inflammatory cytokine, and inhibiting viral neuraminidase (e.g., the influenza viral neuraminidase) of a cell found within a body of a living organism. Embodiments of the present disclosure provide phytochemical compositions that include predetermined concentrations of sesamin for at least one of decreasing gene expression of a pro-inflammatory cytokine, decreasing quantity of a pro-inflammatory cytokine, increasing gene expression of an anti-inflammatory cytokine, and increasing quantity of an anti-inflammatory cytokine released from a cell found within the body of the living organism.

The reduction in quantity of the pro-inflammatory cytokine, increase in quantity of the anti-inflammatory cytokine, and/or inhibition of viral neuraminidase within the body of the living organism facilitates at least one of prevention, control, down-regulation, or termination of cytokine storms or un-regulated inflammation within the body of the living organism. In several embodiments, phytochemical compositions provided by the present disclosure facilitates at least one of prevention, control, down-regulation, or termination of cytokine storms or inflammation caused or triggered by viral infections (e.g., infections by influenza viruses such as influenza A (H1N1), influenza A (H2N1), influenza A (H2N2), and influenza A (H2N3) viruses). In some embodiments, phytochemical compositions provided by the present disclosure have anti-viral and/or anti-inflammatory properties. In specific embodiments, particular phytochemical compositions provided by the present disclosure are effective for reducing or beneficially modulating physical effects (e.g., tissue damage) caused at least partly by occurrences of cytokine storms within the body.

In specific embodiments, particular phytochemical compositions provided by the present disclosure are effective against various infectious and non-infectious diseases, such as influenza (e.g., influenza A) and ARDS. In selected embodiments, particular phytochemical compositions provided by the present disclosure are effective for reducing or beneficially modulating inflammation or tissue degradation produced in association with infectious and non-infectious diseases or conditions (e.g., Osteoarthritis, Osteoporosis).

Uses of sesamin for the manufacture of particular phytochemical compositions as disclosed herein are also provided by the present disclosure. In some embodiments of the present disclosure, the phytochemical composition is a food product (e.g., is manufactured primarily as a food product). In other embodiments, the phytochemical composition is a beverage product (e.g., is manufactured primarily as a beverage product). In yet other embodiments, the phytochemical composition is a supplement composition for one of a food product and a beverage product. In some embodiments, the phytochemical composition is a drug or pharmaceutical product. In other embodiments of the present disclosure, the phytochemical composition is a supplement, additive, or ingredient for one of a food product, a beverage product, and a drug, pharmaceutical, or other consumable (e.g., ingestible) product. It will be understood by a person having ordinary skill in the art that the phytochemical composition can be in various forms or formulations, for example a powder, a paste, or an emulsion.

Although embodiments and specific examples and experiments of the present disclosure are described above, the present disclosure is not to be limited to specific details so described. The scope of the present disclosure is not limited to the embodiments of the present disclosure provided above. A person having ordinary skill in the art will understand that numerous changes and modifications can be made to the embodiments, examples, and experiments without departing from the scope or spirit of the present disclosure.

1-19. (canceled)

20. A method of reducing inflammation in connective tissue of a living organism comprising contacting the connective tissue with a composition of sesamin and glucosamine compound in a molar ratio of 0.0625-1:5-80.

21. The method of claim 20, wherein the connective tissue is at least one of a dense connective tissue, a fibrous connective tissue, a cartilage and a bone.

22. The method of claim 20, wherein the glucosamine compound is at least one of glucosamine sulfate and glucosamine hydrochloride.

23. The method as in claim 20, wherein at least 5 mg of sesamin is administered to the living organism.

24. The method as in claim 20, wherein the living organism is a human being.

25. The method as in claim 20, wherein the composition is administered to the living organism in the form of at least one of a supplement, an additive in a food, a beverage, a pharmaceutical composition, and a phytochemical composition.

26. The method claim 20, wherein the method results in the a decrease in the concentration of hyaluronic acid in at least one of the cartilage, an extracellular matrix and a synovial fluid.

27. The method as in claim 20, wherein the composition is co-administered to the living organism with at least one substance selected from the group consisting of an anti-inflammatory substance and a cholesterol lowering substance, wherein the anti-inflammatory substance is selected from at least one of a chondroitin compound and an omega-3 fatty acid and the cholesterol lowering substance is a statin.
